(19) United States
(12) Patent Application Publication
(10) Pub. No.: US 2022/0402895 A1
(43) Pub. Date: Dec. 22, 2022

(54) Method and System for Synthesising Compounds

(71) Applicant: National University of Singapore, Singapore (SG)

(72) Inventors: Jie WU, Singapore (SG); Saif A. KHAN, Singapore (SG); Chenguang LIU, Singapore (SG); Wenbin WU, Singapore (SG); Weihao CHEN, Singapore (SG); Mu WANG, Singapore (SG); Jiaxun XIE, Singapore (SG)

(21) Appl. No.: 17/770,347

(22) PCT Filed: Oct. 22, 2020
(86) PCT No.: PCT/SG2020/050603

§ 371 Date: Apr. 20, 2022

Related U.S. Application Data
(60) Provisional application No. 62/925,031, filed on Oct. 23, 2019.

(51) Int. Cl. C07D 403/12; B01J 8/02

(52) U.S. Cl. CPC C07D 403/12; B01J 8/02; B01J 2219/00162; B01J 2219/00051; B01J 2219/00164

(57) ABSTRACT

The present invention relates, in general terms, to a method of synthesising compounds and a system for synthesising compounds. The method and system can be automated. The method of synthesising a compound includes providing a solid support in fluid communication with a computer-controlled flow system, covalently bonding a first precursor to the solid support, performing at least one reaction, and cleaving the compound from the solid support.

### TECHNICAL FIELD

The present invention relates, in general terms, to a method of synthesising compounds and a system for synthesising compounds. The method and system can be automated.

### BACKGROUND

Multidimensional diversification for hit-to-lead optimization for the selection of clinical candidates represents an integral part of pharmaceutical drug discovery. Exploring diverse chemical space starting from a potential lead compound is a routine practice in medicinal chemistry to understand structure-activity relationships (SAR), optimize the on-target potency, and improve the absorption, distribution, metabolism, and excretion (ADME) properties. However, experimental synthesis is a labor-intensive undertaking, as chemists must invest substantial time and money in repetitive reaction manipulation and downstream purification. In this regard, late-stage functionalization is particularly appealing since it can provide a large panel of analogue scaffolds based on a lead compound without resorting to multi-step de novo syntheses. In stark contrast, diversification through early-stage functionalization has seldom been approached due to the cumbersome nature of such efforts.

Compared to the batch-wise step-by-step synthesis, multi-step continuous flow synthesis enables the combination of several synthetic steps into a single and uninterrupted reactor network, thereby circumventing the need to isolate intermediate products and thus achieving an automatic synthesis. In flow chemistry, a chemical reaction is run in a continuously flowing stream rather than in batch production. If these fluids are reactive, a reaction takes place. The transition from cumbersome manual synthesis to the automated assembly of pharmaceutical molecules has allowed improvements in efficiency, scalability, safety, and reproducibility, and is therefore of widespread academic, industrial, and societal interest. However, other than the well-defined methods for automated peptide and oligonucleotide synthesis (and increasingly oligosaccharides), in which the molecules are composed of repeating functional units, the synthesis of small-molecule-based active pharmaceutical ingredients (APIs) remains predominantly a manual process due to the structural diversity.

In particular, for multistep continuous-flow synthesis, there are currently two models. The first mimics the flow process that is already commonly used in the petroleum industry (FIG. 1ii). Phase-separators have been applied to achieve effective inline separation. However, the solvent/reagent incompatibility between each step, the requirement of numerous pumps, and the build-up pressure of reactors limit the maximum number of sequential reaction steps in flow.

Another method relates to the use of supported reagents and scavenger resins in multistep syntheses (Figure liii). In this approach, the reaction mixture can be passed over polymer-immobilized reagents or scavengers after intermediate steps, thereby avoiding interference of the unnecessary reagents with subsequent reaction steps. However, the downside of this method is the need to efficiently prepare and regenerate reagent and scavenger columns time after time, which eventually make the process less effective.

There are currently many difficulties with continuous flow synthesis. For instance, some considerations are solvent and reagent incompatibility between individual steps, build-up pressure of reactors, substrate dispersion, and requirement of regeneration of reagent and scavenger columns, limit the maximum number of sequential steps in flow, the accumulation of side-products, risk of clogging, and mismatch of time scales between steps in a processing chain.

It would be desirable to overcome or ameliorate at least one of the above-described problems, or at least to provide a useful alternative.

### SUMMARY

The present invention is predicated on the understanding that recent advances in the end-to-end continuous-flow synthesis are met with many hurdles that limit the number of sequential steps that can be achieved in such systems; these include solvent and reagent incompatibility between individual steps, cumulated by-product formation, risk of clogging, and mismatch of time scales between steps in a processing chain. In all reported continuous-flow API syntheses, the target molecule grows through sequential transformations in a mobile fashion.

The inventors have found that by merging solid-phase synthesis (SPS) and continuous-flow operation, and this enables push-button automated multistep syntheses of active pharmaceutical ingredients. There are however other issues that must be overcome to provide a streamlined solution. To this end, the present invention is advantageous as there is no limitation on the maximum number of sequential steps in the method. Further, fewer pumps are needed for the system. Further advantageously, the method minimises or eliminates reagent/solvent incompatibility and minimizes infrastructure. Additionally, while prior art examples can only performed “late-stage” functionalization of a lead compound, the presently disclosed invention can produce both “early-stage” and “late-stage” modification on a lead compound. For example, a streamlined, six-step synthesis of prexasertib in 65% isolated yield after 32 hours of continuous execution is shown herein. Further, since there are no interactions between individual synthetic steps in the sequence, the established chemical recipe file (CRF) was directly adopted or slightly modified for the synthesis of twenty-three prexasertib derivatives, enabling both automated late- and early-stage diversification. The flexible CRF, the compact and universal reactor system, together with the rich diversity of available synthetic methods and chemical feedstocks, make this invention an important step towards the goal of on-demand molecular synthesis and enable multistep automation with a much wider range of reaction conditions and reaction types.

Accordingly, the present invention provides a method of synthesising a compound, including:
a) providing a solid support in communication with a computer-controlled flow system;
b) covalently bonding a first precursor to the solid support for forming an extension thereon;
c) performing at least one reaction, the reaction including:
   i) conditioning the extension with a first solvent;
   ii) reacting a first reactant with the extension for synthesising the compound on the solid support; and
d) cleaving the compound from the solid support;
   wherein the first precursor, the first solvent and the first reactant are deliverable to the substrate via the computer-controlled flow system.

In some embodiments, the first reactant is provided in the first solvent.

In some embodiments, the method further includes a step (ciii) after step (cii) of washing the compound with the first solvent.

In some embodiments, the method further includes a step of purging with air or gas before the conditioning step (ci).

In some embodiments, the method further includes a step of purging with air or gas after the washing step (ciii).

In some embodiments, the method further includes a step of altering a temperature of the reaction.

In some embodiments, the solid support is a 2-chlorotrityl chloride resin.

In some embodiments, the first precursor comprises an amino moiety.

In some embodiments, the at least one reaction is a SN₂ reaction, hydrazine condensation, Thorpe reaction, Claisen condensation, S_{N}Ar reaction, amide coupling, N-triflation, reductive amination, phenyl-hydrazine cyclization, pyazole cyclization, click chemistry, or Mitsunobu reaction.

In some embodiments, the first precursor further comprises a second moiety selected from the group consisting of halide (except fluoride), tosylate, amino, carboxyl, carbonyl, triflate, aryl, alkynyl, azidyl, alkenyl, tetrazinyl, tetrazolyl, hydroxyl, hydrazoic acid, imide, thiophenol, sulphonamide, arylsulfonylhyrazine, hydrazine, or cyanoalkylacyl.

In some embodiments, the compound is cleaved using trifluoroacetic acid.

The present invention also provides a method of synthesising prexasertib, derivatives, salts, solvates or stereoisomers thereof, comprising:
a) providing a 2-chlorotrityl chloride resin in communication with a computer-controlled flow system;
b) covalently bonding a compound of Formula (I) to the 2-chlorotrityl chloride resin for forming an extension thereon, the compound of Formula (I) is:

T-M-NHR₁ (I)
wherein M is optionally substituted alkyl;
T is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₁ is selected from H, optionally substituted alkyl, optionally substituted alkenyl;
c) performing a first reaction, the first reaction including:
i) conditioning the extension with a first solvent;
ii) reacting a compound of Formula (II) with the extension for synthesising a first intermediate on the solid support, the compound of Formula (II) is:

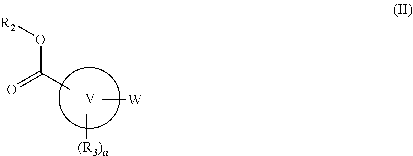

wherein V is selected from optionally substituted aryl or optionally substituted heteroaryl;
W is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₂ is optionally substituted alkyl;
R₃ is independently selected from halide, optionally substituted alkyl, optionally substituted alkenyl, optionally substituted alkoxy;
a is an integer selected from 0, 1, 2 or 3;
d) performing a second reaction, the second reaction including:
i) conditioning the first intermediate with a second solvent;
ii) reacting CH₃CN and lithium diisopropylamide with the first intermediate for synthesising a second intermediate on the solid support;
e) performing a third reaction, the third reaction including:
i) conditioning the second intermediate with a third solvent;
ii) reacting NH₂NH₂ or NH₂NHPh with the second intermediate for synthesising a third intermediate on the solid support;
f) performing a fourth reaction, the fourth reaction including:
i) conditioning the third intermediate with a fourth solvent;
ii) reacting a compound of Formula (III) with the third intermediate for synthesising a fourth intermediate on the solid support, the compound of Formula (III) is:

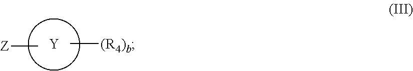

wherein Y is selected from optionally substituted aryl, optionally substituted heteroaryl, optionally substituted cycloalkyl, or optionally substituted heterocycloalkyl;
Z is selected from carboxyl, carbonyl, alkenylacyl, triflate, aryl, alkyl (substituted with carboxyl, carbonyl or alkenylacyl), or alkoxy (substituted with carboxyl, carbonyl or alkenylacyl);
R₄ is selected from halo, cyano, optionally substituted alkoxy, optionally substituted alkyl, or optionally substituted alkenyl;
b is an integer selected from 0, 1, 2, or 3; and
g) cleaving the compound from the solid support;
   wherein the compounds of Formula (I), (II), (II), CH₃CN, lithium diisopropylamide, NH₂NH₂ and solvents are deliverable to the 2-chlorotrityl chloride resin via the computer-controlled flow system.

In some embodiments, when the compound synthesised is prexasertib, a yield is at least 60%.

In some embodiments, when the compound synthesised is prexasertib, a purity is at least 99%.

The present invention also provides a system for synthesising a compound, including:
a) a flow system;
b) a solid support in fluid communication with the flow system;
c) a first precursor covalently bonded to the solid support for forming an extension thereon;
d) at least a first solvent for conditioning the extension;
e) at least a first reactant for performing at least one reaction on the extension to synthesise the compound; and
f) a controller configured to regulate the flow system for flowing the first precursor, the first solvent and the first reactant to the substrate.

The solid support may be contained in a reaction vessel; and the flow system may be connected to the reaction vessel for fluid communication with an interior thereof.

In some embodiments, the flow system includes at least one multi-port input valve in communication with the controller, and the controller is configured to actuate the at least one multi-port input valve for selective flow of the first precursor, the first solvent or the first reactant to the solid support.

In some embodiments, the flow system includes a multi-port output valve, and the controller is configured to actuate the multi-port output valve for selective flow of output fluid to one of a plurality of output vessels.

In some embodiments, the flow system includes a pressure regulator for regulating pressure inside the reaction vessel.

In some embodiments, the controller is configured to alter the temperature of the reaction.

The system may further include at least one heating element in thermal communication with the reaction vessel.

In some embodiments, the flow system further includes a circulative flow path for allowing the flow to return to a starting bottle. In other embodiments, the flow system further includes a one-way flow path for allowing the flow to elute to a waste bottle.

### BRIEF DESCRIPTION OF THE DRAWINGS

Embodiments of the present invention will now be described, by way of non-limiting example, with reference to the drawings in which:

FIG. 1 illustrates prior art examples of multistep synthesis of compounds;

FIG. 2 is a schematic illustration of an embodiment of a system for synthesising compounds, which provides a push button approach to synthesising active pharmaceutical ingredients (APIs);

FIG. 3 is a block diagram of the embodiment of FIG. 2;

FIG. 4 is a photo of one embodiment of a system for synthesising compounds, together with a schematic showing various components of the system;

FIG. 5 illustrates a comparative solid-phase synthesis of prexasertib in batch reactors; and

FIG. 6 illustrates the synthesis route of prexasertib using an embodiment of the present invention (automated SPS-based continuous flow platform);

FIG. 7 shows an exemplary development of SPS-flow synthesis of prexasertib. a) Solution-batch synthesis of prexasertib. b) Schematic of the CRF; and

FIG. 8 is a workflow for the development of a chemical recipe file (CRF) for the synthesis of APIs and analogs.

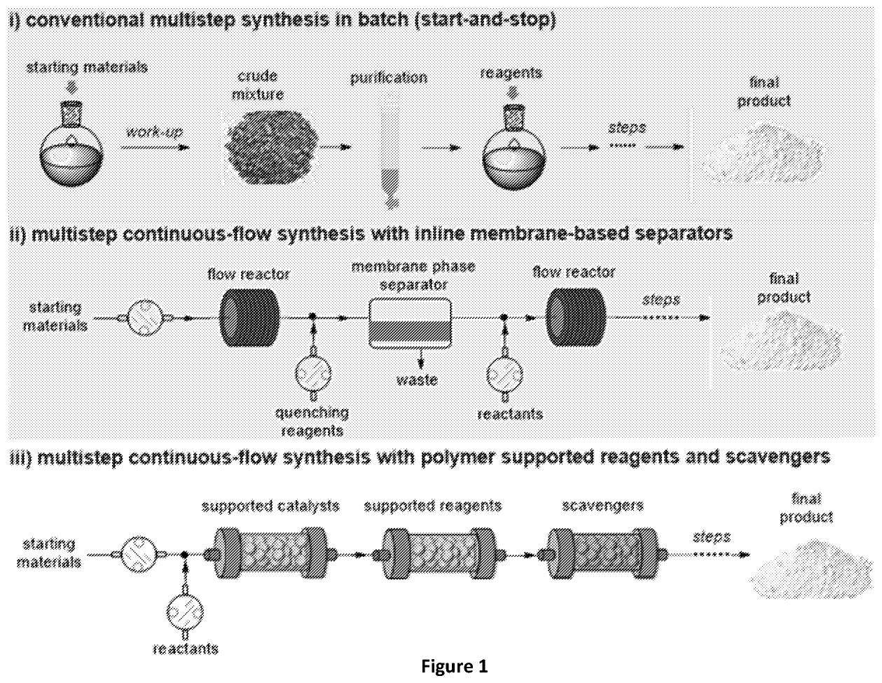

### DETAILED DESCRIPTION

The inventors have studied previous technologies such as iterative deprotection-coupling-purification sequences, robotic systems driven by a chemical programming language, end-to-end on-demand continuous-flow synthesis, and radial synthesis for the automated synthesis of APIs. Compared to conventional stirred reactor vessels, continuous-flow reactors have several significant processing advantages, which include improved mass/heat transfer, enhanced mixing efficiency, better reproducibility, improved safety, reduced footprint, and facile scalability. Multistep continuous-flow syntheses also enable the telescoping of several steps into a single and uninterrupted synthetic process, which circumvents the need to purify and isolate intermediates. However, it is worth noting that fully continuous-flow syntheses rarely exceed two steps before offline purification.

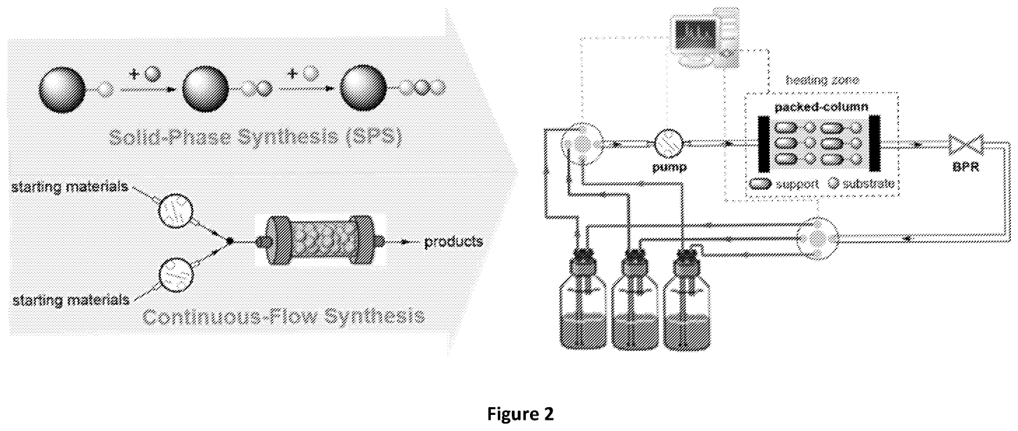

The inventors have found that the synthesis of non-peptide APIs (or small molecule synthesis) can be obtained through the merger of the fundamental concepts in solid phase synthesis (SPS) and continuous-flow synthesis (FIG. 2), i.e. by having the target molecule grow on a solid support while all reagents and catalysts stay in the mobile phase, and the product detached from the resin at the last step followed by a single purification to afford the pure product. The present invention avoids the problem of reagent/solvent/byproduct incompatibility between synthetic steps and enables automation with a much wider range of reaction conditions and reaction types. The synthesis can further be automated.

In SPS, molecules are bound on a polystyrene bead and synthesized step-by-step in reactant solutions. Compared with solution-based synthesis, the key advantage of SPS is that it circumvents tedious intermediate isolation and purification procedures such as recrystallization, distillation, and chromatography, and instead uses simple filtration. To this end, the inventors believed that SPS can be used in combination with continuous flow synthesis to simplify the purification processes of multi-step synthesis.

However, a combination of SPS and continuous flow synthesis is not simple and straightforward. For example, solvent incompatibility when synthesising organic compounds is an issue. When not properly purified, residues can form by-products with reagents downstream which lowers the product yield. Further and in particular, the generation of a computer-based chemical recipe file (CRF) for a target molecule on a standardized platform enables pharmaceutical production in response to sudden changes in demand or need, such as in epidemic or pandemic instances of influenza outbreak. However, establishing such a CRF using SPS-based continuous-flow technology requires several stages of development Small molecules often possess inherent complexity associated with their molecular framework; as such, translating batch syntheses into automated liquid-phase continuous-flow syntheses can be challenging, often requiring new synthetic routes or new reagents and advanced purification techniques. Moreover, the number and sequence of units in a continuous processing cascade are tailored to match a specific synthetic route, and it is difficult to reconfigure a system constructed for a specific molecule for another target. This limits the widespread adoption of automated flow syntheses, as one may question whether the convenience afforded by automation of a specific target is worth the substantial efforts required to develop such a protocol.

The inventors have found that the translation of solution-batch synthesis to SPS-flow synthesis (i.e. by controlling the flow instead of using continuous flow) is much more straightforward for several reasons. First, each step is performed independently, thus avoiding reagent/solvent/by-product incompatibility issues. Next, unlike liquid-phase continuous-flow synthesis, there is no limitation on reaction time in SPS-flow synthesis, and mismatched time-scales between subsequent steps do not affect performance. Third, the same system hardware can be reused for many targets without any physical reconfiguration. Finally, clogging is less problematic as a column reactor with a large threshold filter can be applied. SPS-flow synthesis of the present invention can significantly reduce the complexity, cost, and footprint of infrastructure needed, and dramatically simplify and accelerate the translation of solution-batch syntheses to automated SPS-flow CRFs. The established CRFs can be directly adopted or slightly modified for the synthesis of lead analogues, allowing both late- and early-stage diversification. End-to-end API synthesis can be realized with no limitation on the maximum number of sequential steps. More importantly, it will enable an automated early-stage derivatization of drug molecules as well as late-stage derivatization.

Further, all the reaction procedures can be controlled and monitored by computer (e.g. one or more microcontrollers) to achieve automated synthesis. The starting material (precursor) can be immobilized on the resins in a packed column, followed by pumping of reagents through the column step by step in an appropriate sequence. In this manner, all intermediate reagents are readily removed (unreacted reagents, side products and waste) at the completion of each step, and the accumulation of reagents is avoided, which bypasses incompatibility problems. So any step can be altered or replaced by other kinds, rendering early- and late stage functionalization. Besides, the system has significant processing advantages, which include improved mass/heat transfer, enhanced mixing control, less footprint requirement, and can be safer than batch reactors. The flexibility of this reactor system allows for a wide range of applications, which would be very marketable to both academia and chemical engineering industry.

Analysis of the top 200 small-molecule pharmaceuticals (by retail sales in 2008), the inventors envision that 73% of the single pharmaceutical molecules among them can potentially be synthesized by the presently disclosed method and system.

Advantageously, there is no limitation on the maximum number of sequential steps in the method as disclosed herein, and fewer pumps can be applied to the system. Reagent/solvent incompatibility can be minimised, as with the infrastructure. In this regard, the inventors have found that by conditioning the resin (substrate) with an appropriate solvent, the previous and incompatible solvent can be washed out while at the same time preparing the resin for the next reaction. Further, the disclosed method can produce both early-stage and late-stage modification products (FIG. 2).

The present invention has the following further advantages:

Polystyrene resin can be used as solid support material: The products and synthetic intermediates can be separated and isolated easily since all substrates are immobilized on polystyrene resin. Tedious intermediate isolation and purification procedures such as recrystallization, distillation, and chromatography can be circumvented;

Merging solid-phase synthesis (SPS) and multi-step flow synthesis: Reagents with high activity, such as BuLi, or reagents with low boiling points can be easily handled in flow system. Reactions may be carried out at temperatures higher than the boiling points of the solvent and reagents in atmospheric pressure. The system can reduce the infrastructure and the amount of resins and columns, in contrast to traditional solid-phase synthesis. Any change of reagents or solvents at any stage of the synthesis would not interfere with the following step. The synthetic procedure can be easily scaled up to kilogram grade with less space requirement.

Automated control by connecting to a computer: By connecting to a computer system, the concept “push button approach for API synthesis” can be achieved. The whole synthetic process can be fully automated. Both automated “early” and “late stage” derivatization can be realised, which is advantageous as an efficient method for “early stage” derivatization has never been achieved til now. This opens up the possibility of utilising more diverse chemical reactions. Further, the automation allows for real time monitoring of the reaction progress, for example by connecting to a mass spectrometer.

Other advantages of the present invention (FIG. 2) includes:
A push button approach for API synthesis
Fully automated and on demand
No reagent/solvent incompatibility
Minimised infrastructure
Scalable
Reduced risk of clogging
Automated derivative synthesis

Accordingly, the present invention provides a method of synthesising a compound, including:
a) providing a solid support in fluid communication with a computer-controlled flow system;
b) covalently bonding a first precursor to the solid support for forming an extension thereon;
c) performing at least one reaction, the reaction including:
   i) conditioning the extension with a first solvent;
   ii) reacting a first reactant with the extension for synthesising the compound on the solid support; and
d) cleaving the compound from the solid support;
   wherein the first precursor, the first solvent and the first reactant are deliverable to the solid support via the computer-controlled flow system.

In some embodiments, the method of synthesising a compound is a method of synthesising a small molecule compound. A small molecule is a low molecular weight (<900 daltons) organic compound that may regulate a biological process, with a size on the order of 1 nm. Peptide and proteins are excluded from this definition.

The method for coupling reactions can be performed in the presence of various solvents at different optimised conditions. Such solvents and conditions are often not compatible with each other. In stark contrast, peptide synthesis performed using SPS is often performed in water as the only solvent and only for amide coupling.

As used herein, ‘solid support’ refers to a non-soluble material, the surface of which can be used to synthesise a compound. In this regard, the solid support can be a polymeric resin bead. A solid support can be made up of materials such as polystyrene, polystyrene-PEG composites, PEG and poly-ε-lysine (ε-PL). Such a solid support can be functionalized with reactive groups (such as amine or hydroxyl groups). The solid support can be characterised by its loading level and swelling capacity in organic solvents. In this regard, the physical aspects of the solid support such as composition, bead size, and chemical aspects such as functionalisation can influence its use and downstream chemistry.

As used herein, “extension” refers at least to the first precursor that is covalently bonded to the solid support. The extension can grow in the sense that a reactant can react with a functional group on the extension and add on it. In this sense, the extension is prolonged or enlarged by one or more reactions with one or more reactants.

As used herein, “conditioning” includes a step of washing the solid support with a solvent. This washing step is preferably done before the reaction step. The solvent can be a solvent that is the same or similar to that used in the reaction. For example, if DMF is used in the reaction, the conditioning step includes washing the solid support with DMF, after which the reaction on the solid support is performed in DMF.

In some embodiments, the compound to be synthesized has an amino moiety. As used herein, “amino” refers to the group —NR″R″ where each R″ is independently hydrogen, alkyl, cycloalkyl, aryl, heteroaryl, and heterocyclyl.

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Halo” or “halogen” or “halide” refers to fluoro, chloro, bromo and iodo.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronapthalenyl and the like.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like. “Heteroaryl” refers to a monovalent aromatic heterocyclic group which fulfils the Hückel criteria for aromaticity (ie. contains 4n+2 π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Acyl” refers to groups H—C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl are as described herein.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein. An example is carboxyl.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like.

In some embodiments, the solid support is a polystyrene bead. In other embodiments, the solid support is a 2-chlorotrityl chloride resin.

In some embodiments, the first precursor comprises an amino moiety. In other embodiments, the first precursor comprises an ammonium halide moiety (—NH₂.HX). The halide can be fluoride, chloride, bromide or iodide.

In some embodiment, when the solid support is a 2-chlorotrityl chloride resin and the first precursor comprises an ammonium halide moiety. The first precursor can be covalently bonded to the solid support via a S_{N}1 or S_{N}2 reaction. The reaction can be performed in the presence of a base, such as triethylamine.

In some embodiments, the first precursor further comprises a second moiety. The second moiety allows a subsequent reaction to take place on the precursor in order to form the compound. When the reaction is an SN₂ reaction, the second moiety can be halide (except fluoride) or tosylate. When the reaction is an amide coupling, the second moiety can be amino, or carboxyl group. When the reaction is a reductive amination, the second moiety can be a carbonyl group. When the reaction is a triflation reaction, the second moiety can be triflate, amine, or hydroxyl. When the reaction is a S_{N}Ar (nucleophilic aromatic substitution) reaction, the second moiety can be aryl. When the reaction is a click reaction, the second moiety can be alkynyl, azidyl, alkenyl, tetrazinyl, or tetrazolyl. When the reaction is a Mitsunobu reaction, the second moiety can be hydroxyl, carboxyl, hydrazoic acid, imide, phenol, thiophenol, sulphonamide, or arylsulfonylhyrazine. When the reaction is a pyazole cyclization, the second moiety can be hydrazine, carbonyl, or cyanoalkylacyl.

For example, when the reaction is reductive amination, a pyrazole-amine can be reacted with 2-formylofuran at 80° C. in EtOH-THF for 3 hours. After washing, the resulted imine product was subjected to LiAlH₄ in THF at room temperature to furnish the reductive amination product.

In some embodiments, the first reactant is provided in the first solvent.

In some embodiments, the solvent is an aqueous medium. The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

In other embodiments, the solvent is organic medium. As used herein, the term “organic medium” is an organic based solvent or solvent system, and which comprises of mainly organic solvent. Organic based solvents can be any carbon based solvents. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Organic based solvents or solvent systems can include, but not limited to, any non-polar liquid which can be hydrophobic and/or lipophilic. As such, oils such as animal oil, vegetable oil, petrochemical oil, and other synthetic oils are also included within this definition.

In some embodiments, the step of performing at least one reaction further includes a step of washing the solid support. In other embodiments, the step of performing at least one reaction further includes a step after step (cii) of washing the compound with the first solvent.

In some embodiments, the step of performing at least one reaction further includes a step of altering a temperature of the reaction. The temperature can be altered from about 15° C. to about 130° C.

In some embodiments, the step of performing at least one reaction further includes a step of filtering the solid support. This can be done using a frit disc. Advantageously, the frit disc prevents the resin inside the column from being washed out by the reagent flow.

In some embodiments, the step of performing at least one reaction further includes a step of purging the solid support with air or gas. The gas can be argon, neon, helium, hydrogen, nitrogen, oxygen, carbon monoxide, carbon dioxide, or a combination thereof. In other embodiments, the step of performing at least one reaction further including a step of purging with argon before the conditioning step (ci). In other embodiments, the step of performing at least one reaction further including a step of purging with argon after the washing step (ciii).

Further advantageously, it was found that a purge with an inert gas such as argon pushes out excess and/or unreacted reagents from the system such that the subsequent reaction can be facilitated. In this way, transiting between immiscible solvents for different reactions is possible. The inventors have found that this purging step is advantageous over a solvent evaporation step, for example using a rotary evaporator, as it further reduces the loss of intermediates/compounds.

In some embodiments, the step of performing at least one reaction further includes a step of washing with a co-solvent. Co-solvents are substances added to a primary solvent to increase the solubility of a poorly-soluble compound or another solvent. This step can be done after the washing step with the first solvent or after the purging step.

In some embodiments, the step of performing at least one reaction further includes a step of purging the solid support with air or gas after the step of washing with a co-solvent.

Advantageously, purging with air or gas and/or washing with a solvent and/or co-solvent removes or at least minimises the interaction between individual steps. To this end, by-products from residual reagents can be eliminated or at least minimised.

In some embodiments, the at least one reaction is a SN₂ reaction, hydrazine condensation, Thorpe reaction, Claisen condensation, S_{N}Ar reaction, amide coupling, triflation, reductive amination, phenyl-hydrazine cyclization, pyazole cyclization, click chemistry, or Mitsunobu reaction.

In some embodiments, the Claisen condensation is performed at room temperature, or about 15° C. to about 30° C. In other embodiments, the Claisen condensation is performed using lithiated acetonitrile. In other embodiments, the Claisen condensation is performed for about 30 min.

In some embodiments, the compound is cleaved using trifluoroacetic acid. In other embodiments, the compound is cleaved in the presence of CH₂Cl₂. In other embodiments, the compound is cleaved at room temperature, or about 15° C. to about 30° C.

In some embodiments, the compound can be obtained without flash chromatography.

In some embodiments, the method allows for a linear synthesis of a compound. In this regard, the method can, for example, utilise one solid support column. In other embodiments, the method allows for a convergent synthesis of a compound. In this regard, the method can, for example, comprise more than one solid support column, the product of which can come together to form an end product.

In some embodiments, the method further comprises a step of crystallising the compound. The purity of the compound can be more than 90%, 92%, 94%, 96%, 98%, 99%, or 99.5%.

The advantages of the present invention are that it is particularly applicable to specific types of reactions (e.g. SN₂, Claisen condensation, Mitsunobu, etc), and enantiopure forms of compounds can be obtained. Further, as pharmaceutically acceptable reagents and solvents are used and the reactants can be easily washed away, an end product with a higher purity can be obtained. The present invention also provides more cost savings and is green as unreacted reagents can be recycled back into system. As is shown herein, a higher yield and efficiency is obtained compared to SPS alone, and/or continuous flow synthesis alone.

The present invention is particularly applicable to the synthesis of prexasertib.

The present invention also provides a method of synthesising prexasertib, derivatives, salts, solvates or stereoisomers thereof, comprising:
a) providing a 2-chlorotrityl chloride resin in communication with a computer-controlled flow system;
b) covalently bonding a compound of Formula (I) to the 2-chlorotrityl chloride resin for forming an extension thereon, the compound of Formula (I) is:

T-M-NHR₁ (I)
wherein M is optionally substituted alkyl;
T is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₁ is selected from H, optionally substituted alkyl, optionally substituted alkenyl;
c) performing a first reaction, the first reaction including:
i) conditioning the extension with a first solvent;
ii) reacting a compound of Formula (II) with the extension for synthesising a first intermediate on the solid support, the compound of Formula (II) is:

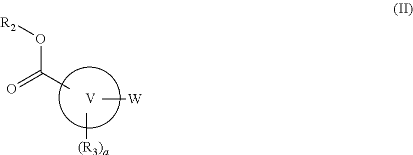

wherein V is selected from optionally substituted aryl or optionally substituted heteroaryl;
W is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₂ is optionally substituted alkyl;
R₃ is independently selected from halide, optionally substituted alkyl, optionally substituted alkenyl, optionally substituted alkoxy;
a is an integer selected from 0, 1, 2 or 3;
d) performing a second reaction, the second reaction including:
i) conditioning the first intermediate with a second solvent;
ii) reacting CH₃CN and lithium diisopropylamide with the first intermediate for synthesising a second intermediate on the solid support;
e) performing a third reaction, the third reaction including:
i) conditioning the second intermediate with a third solvent;
ii) reacting NH₂NH₂ or NH₂NHPh with the second intermediate for synthesising a third intermediate on the solid support;
f) performing a fourth reaction, the fourth reaction including:
i) conditioning the third intermediate with a fourth solvent;
ii) reacting a compound of Formula (III) with the third intermediate for synthesising a fourth intermediate on the solid support, the compound of Formula (III) is:

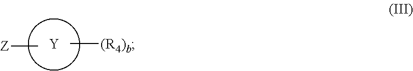

Wherein Y is selected from optionally substituted aryl, optionally substituted heteroaryl, optionally substituted cycloalkyl, or optionally substituted heterocycloalkyl;
Z is selected from carboxyl, carbonyl, alkenylacyl, triflate, aryl, alkyl (substituted with carboxyl, carbonyl or alkenylacyl), or alkoxy (substituted with carboxyl, carbonyl or alkenylacyl);
R₄ is selected from halo, cyano, optionally substituted alkoxy, optionally substituted alkyl, or optionally substituted alkenyl;
b is an integer selected from 0, 1, 2, or 3; and
g) cleaving the compound from the solid support;
   wherein the compounds of Formula (I), (II), (II), CH₃CN, lithium diisopropylamide, NH₂NH₂ and solvents are deliverable to the 2-chlorotrityl chloride resin via the computer-controlled flow system.

In some embodiments, the compound is cleaved with trifluoroacetic acid. The resultant compound is thus eluted as a trifluoroacetate salt form.

In some embodiments, compound of Formula (I) is

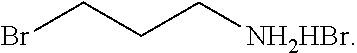

In other embodiments, the compound of Formula (I) is selected from

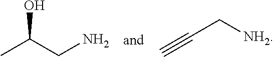

In some embodiments, compound of Formula (II) is

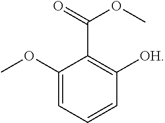

In other embodiments, the compound of Formula (II) is selected from

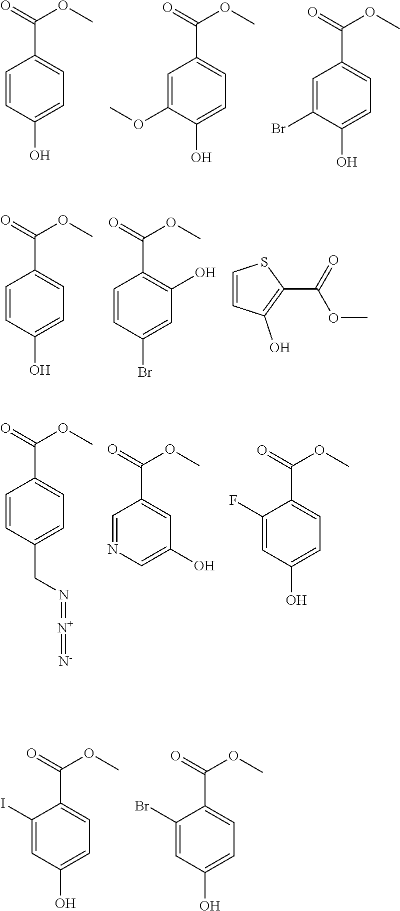

In some embodiments, compound of Formula (III) is

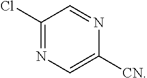

In other embodiments, the compound of Formula (III) is selected from

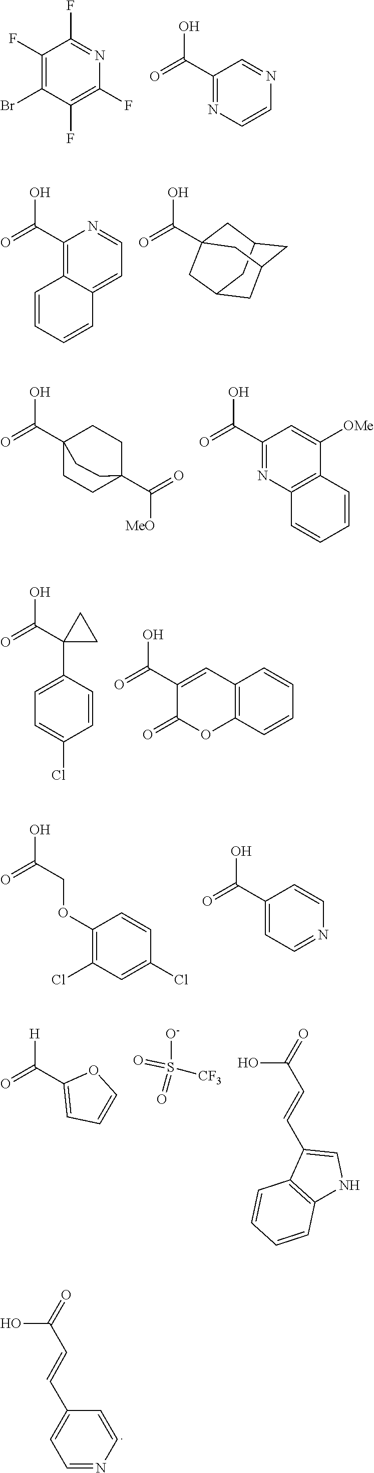

In some embodiments, when the compound synthesised is prexasertib, a yield is at least 60% is obtained. In other embodiments, the yield is at least 50%, 55%, 62%, 64%, 66%, 68%, 70%, 75%, 80%, or 85%.

In some embodiments, when the compound synthesised is prexasertib, a purity is at least 99%. In other embodiments, the purity is at least 99.1%, 99.2%, 99.3%, 99.4%, 99.5%, 99.6%, 99.7%, 99.8%, or 99.9%.

The present invention also provides a system for synthesising a compound, including:
a) a flow system;
b) a solid support in fluid communication with the flow system;
b) a first precursor covalently bonded to the solid support for forming an extension thereon;
c) at least a first solvent for conditioning the extension;
d) at least a first reactant for performing at least one reaction on the extension to synthesise the compound; and
d) a controller configured to regulate the flow system for flowing the first precursor, the first solvent and the first reactant to the solid support.

In some embodiments, the flow system comprises a T-mixer positioned upstream to the solid support in order to mix different reagents and/or solvents. In other embodiments, the mixing by the T-mixer is a homogenous mixing.

In some embodiments, the flow system further comprises a circulative flow path for allowing the flow to return to a starting bottle. In other embodiments, the flow system further comprises a one-way flow path for allowing the flow to elute to a waste bottle.

In some embodiments, the flow system includes a pressure regulator for regulating pressure inside the reaction vessel. In other embodiments, the flow system includes at least one back pressure regulator. The back pressure regulator can be positioned downstream to the solid support and at an outlet to a waste bottle. The back pressure regulator can be positioned downstream to the solid support and along a recycle flow path.

In some embodiments, the flow system further comprises a gas line. Air or gases such as argon, neon, helium, hydrogen, nitrogen, oxygen, carbon monoxide, carbon dioxide can be supplied to the solid support via the gas line.

In some embodiments, the flow system includes at least one multi-port input valve in communication with the controller. The controller can be configured to actuate the at least one multi-port input valve for selective flow of the first precursor, the first solvent or the first reactant into the reaction vessel.

In some embodiments, the flow system includes a or at least one multi-port output valve. The controller can be configured to actuate the multi-port output valve for selective flow of output fluid from the reaction vessel to one of a plurality of output vessels.

In some embodiments, the solid support is fitted with frits disk having a pore size of about 75 μm. In other embodiments, the solid support is fitted with frits disk having a pore size of about 100 mesh. In other embodiments, the solid support is fitted with at least one heating element in thermal communication with the reaction vessel. The heating element can be a heating plate.

In some embodiments, the solid support is contained in a reaction vessel. The reaction vessel can be a column arranged such that the solvent and reactant can fold and pass through substantially all the solid support. In other embodiments, a fluidised bed is used. In some embodiments, the flow system is connected to the reaction vessel for fluid communication with an interior thereof.

In some embodiments, the controller is configured to regulate the flow system in a circulative flow and/or a one-way flow. A circulative flow reverts the flow of solvent and/or reactant back to the solid support. In this way, the flow of solvent and/or reactant passes through the solid support for a plurality of times, or at least twice. A one-way flow pushes the flow of solvent and/or reactant away from the solid support after it has pass through the solid support once.

In some embodiments, the controller is configured to alter the temperature of the reaction. The temperature can be of about 40° C. to about 150° C., about 50° C. to about 150° C., about 50° C. to about 140° C., about 50° C. to about 130° C., or about 50° C. to about 120° C.

The controller may include, or be in communication with, machine-readable storage that stores instructions for causing the system to carry out a multistep synthesis. In this regard, the controller may be programmed in accordance with one or more sequences of steps of the multistep synthesis. Respective steps may include one or more of: actuating an input valve or an output valve of the flow system to cause flow of reagents into and/or out of the reaction vessel; heating the reaction vessel; or regulating the pressure inside the reaction vessel. Respective steps may have respective predetermined durations.

The computer controlled flow system can halt and start a flow as when required. This is advantageous as it allows for the reduction of solvents and reactants used, thus saving cost and making the process more environmentally friendly. Further, manual labour can be reduced for at least the duration of the synthesis. This is advantageous for a synthesis that spans for more than 24 h. Additionally, the flow system is a friendly platform compatible with downstream in-line analysis and purification modules.

The system may further include at least one heating element in thermal communication with the reaction vessel. The heating element can be a heating plate, or can be a heating sleeve.

After the synthesis, the compound is cleavable from the solid support. The compound can thus be eluted out from the flow system, collected and crystallised. Further modifications such as converting the compound to another salt form can also be carried out.

In some embodiments, the compound is synthesizable in a kilogram scale. For example, the compound can be synthesised at a scale of about 1 kg, 2 kg, 3 kg, 5 kg, 10 kg, 15 kg, or 20 kg.

A detailed description of the workings of the invention is laid out below. In the embodiments that follows, the invention is described in relation to prexasertib as an example to showcase the present invention. However, the skilled person would understand that the invention is not limited to such.

The SPS-based flow synthesis platform may include one Vaportec® peristaltic pump and one Syrris® asia-syringe pump for the introduction of different substrate solutions. Different solution injections may be controlled by three VICI® valves and converge with the aid of a T-mixer, and may finally flow into the column consisting of resin. When the mixture is converged and introduced into the column, the reaction starts. After reactions finished, the outflow solution may meet the fourth VICI® valve. According to examples of the designed procedure, there are two pathways for the solution flow: 1) go back to the starting bottle and recycle, or 2) go to the waste solvent bottle. Three back-pressure regulators (BPR) may be located at downstream, near 1) the back flow of the fourth step, 2) the back flow of the fifth step, and 3) the outlet of waste solvent, ensuring that the system is pressurized during the whole synthetic process. The whole system is connected to, or includes, a suitably programmed computer, which allows the platform to achieve the “Push-Button API Synthesis”.

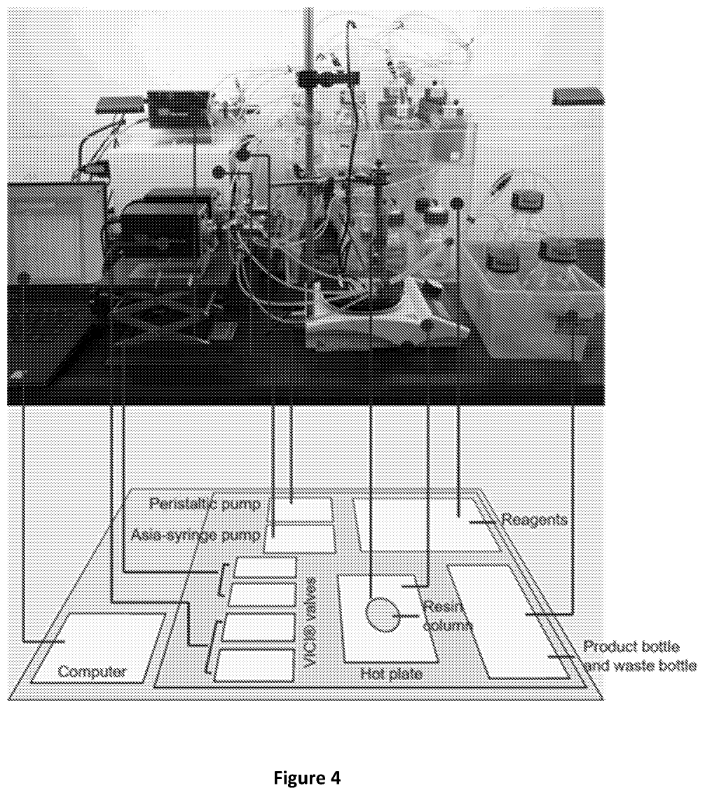

In FIG. 4, the SPS-based flow synthesis platform was assembled. Parts of the reaction system, such as valves, pumps and tubing can be purchased commercially from various sources, and assembled according to the researcher's design, which allows it to be significantly cost-effective and commercial favor. As the traditional solid-phase synthesis requires numerous resins and columns, the new designed system can effectively reduce the number of pumps and the amount of resins and columns. With this platform, reagents with high activity, such as BuLi, or reagents with low boiling points can be easily handled. The protocol can be even scaled up to kilogram scale with reduced footprint. Most importantly, by connecting to a computer system, the whole synthetic process is fully automated, and the concept “push button approach for API synthesis” can be achieved. Moreover, “early” and “late stage” derivatization of APIs can be obtained with slight modifications to the CRF.

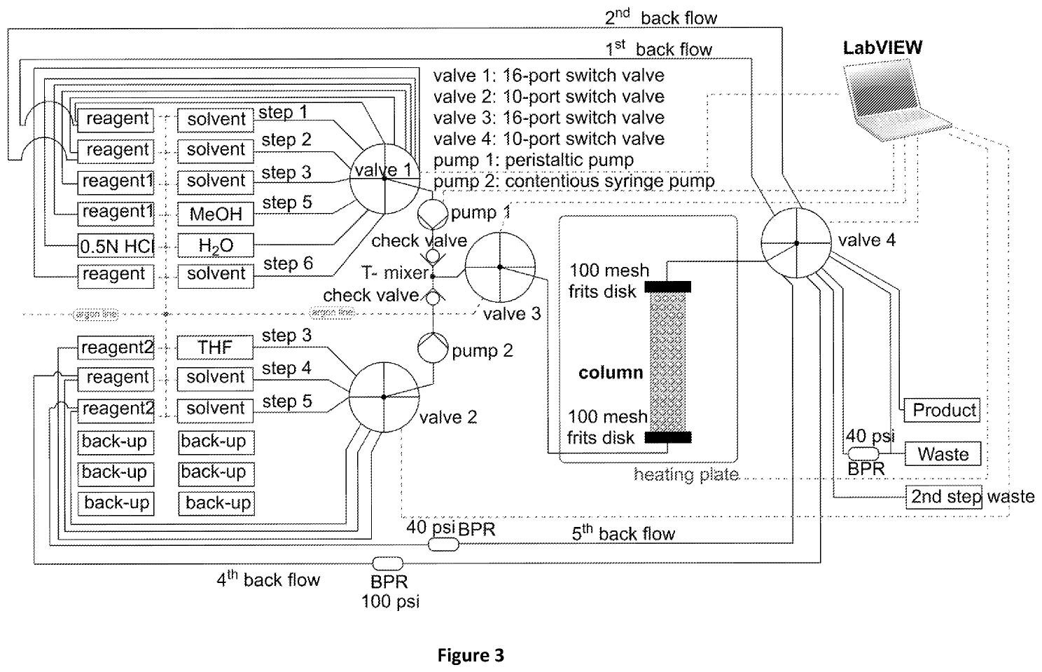

An exemplary modular platform is assembled, consisting of a high-pressure pump equipped with two channels, a peristaltic pump, four multiway selection valves, a stainless-steel column reactor (fitted with frits with 75-μm pores), a digital heating plate, and three back-pressure regulators (BPRs) (FIG. 3). Two of the multiway valves allow the routing of reagents and solvents through the column reactor. The third valve either loops the reactor outflow back into the column reactor or directs it to the end waste or product container. The gas line is controlled by the fourth valve. The whole system can be controlled by a LabVIEW™ interface, which gives a complete description of the connectivity of all units, and facilitates the movement of reagents and solvents from specific stock locations to the column reactor in a circulative or one-way flow fashion. This platform represents one of the simplest and most compact systems for automated multistep synthesis, and it can fit within the area of half a standard fume hood (56 cm (W)×88 cm (L)×56 cm (H), FIG. 4).

As SPS-flow synthesis addresses each synthetic step independently by simple washing and filtration between each step, and the physical hardware can be reused for different types of reactions.

To demonstrate the feasibility of this invention, prexasertib, a small ATP-competitive selective inhibitor of checkpoint kinases 1 and 2 (CHK1 and CHK2) developed by Eli Lilly, was synthesized via a streamlined, six-step synthesis using a compact SPS-flow platform. Twenty-three analogues were prepared through a push-button approach with or without slight adjustments of the established CRF.

The synthesis of Prexasertib can be achieved in a linear fashion with a protection step at the first step. A solid matrix can be applied as a protecting group without introducing additional synthetic steps to anchor the starting material onto the solid matrix. A seven-step synthesis of prexasertib was accomplished by researchers at Eli Lilly, employing a continuous-flow process for the last three steps to achieve a kilogram-scale synthesis. However, the total yield of the synthesis was only about 30%, the entire flow sequence needs monitoring and must be operated manually, the system is also cumbersome to reconfigure for analogue synthesis, and at the development stage, solving the solvent or reagent incapability issue needs much endeavour.

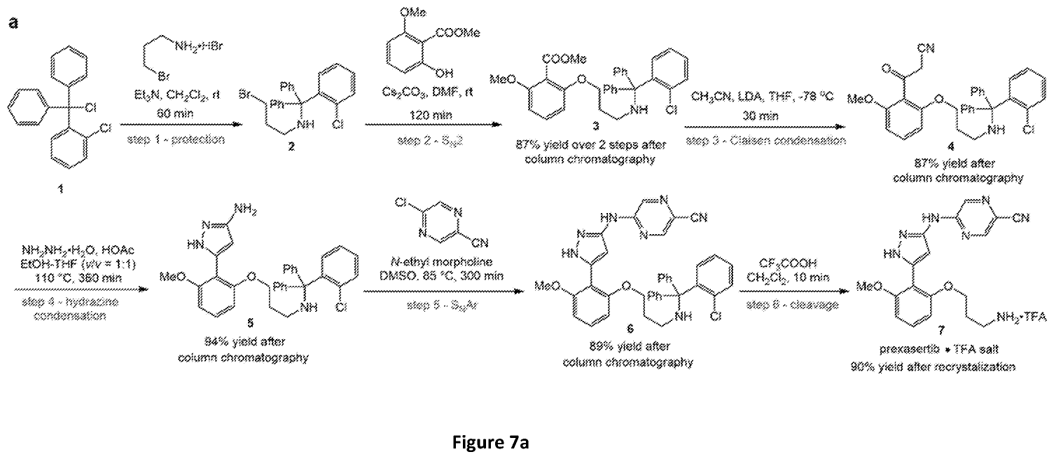

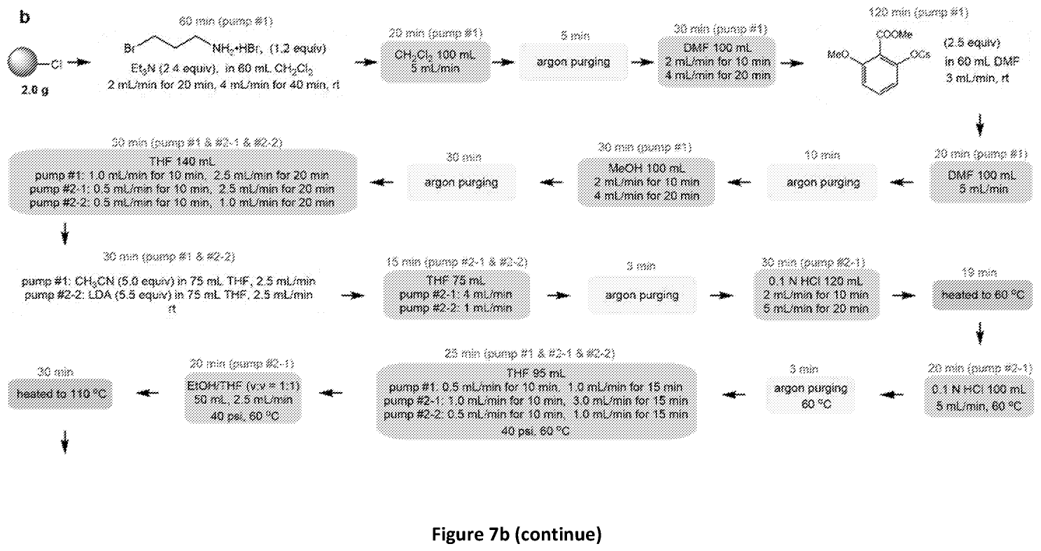

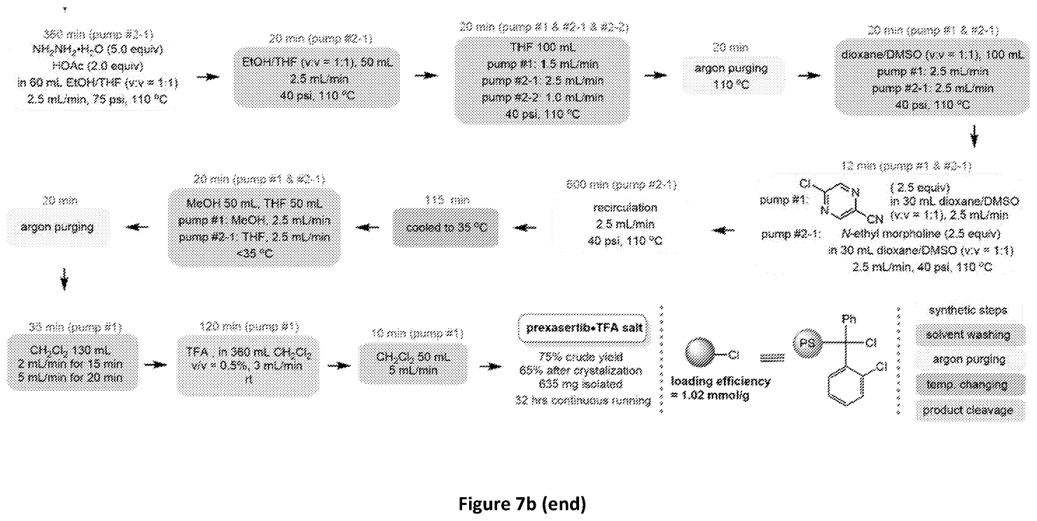

To establish a robust computerized CRF for the automated synthesis of prexasertib, the inventors initiated their investigation with the development of an efficient solution-batch synthesis (FIG. 7; S_{N}Ar=nucleophilic aromatic substitution; DMF=dimethylformamide; LDA=lithium diisopropyl amide; THF=tetrahydrofuran; DMSO=dimethyl sulfoxide; TFA=trifluoroacetic acid; PS=polystyrene resin). The synthesis commenced with protecting 3-bromopropylamine using 2-chlorotrityl chloride (1), which was used to mimic 2-chlorotrityl chloride resin, an inexpensive and commonly utilized resin in SPS. A subsequent S_{N}2 reaction between the substituted phenoxide and primary bromide 2 delivered product 3 bearing an aryl ester moiety. Keto-nitrile intermediate 4 was prepared by a Claisen condensation between aryl ester 3 and lithiated acetonitrile, followed by a hydrazine condensation and an S_{N}Ar. Final removal of the 2-chlorotrityl group by treatment with TFA produced prexasertib as TFA salt 7. The Claisen condensation using lithiated acetonitrile allowed a more straightforward route compared to the Eli Lilly's synthesis. A solution-batch six-step synthesis containing five purification-steps was therefore achieved to afford the TFA salt of prexasertib in 57% overall isolated yield.

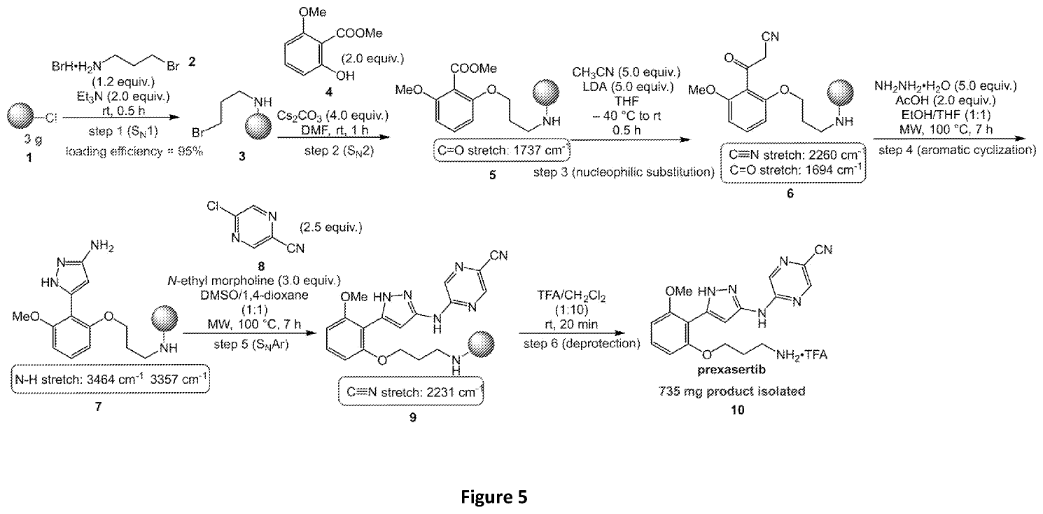

The solution-batch synthesis can be easily translated to the SPS-flow synthesis of the present invention. As shown in FIG. 5, prexasertib was synthesize by solid-phase synthesis in batch. Primary amine was immobilized on the 2-Cl trityl chloride resin (2-Chlorotrityl chloride resin) readily in the presence of Et₃N, with the loading efficiency of 95%. The S_{N}2 reaction proceeded well on the solid phase, delivering intermediate product 5. Then the reaction of the CH₃CN/LDA with product 5 gave product 6 smoothly. The pyazole cyclization also proceeded well in the promotion of HOAc. The pyrazine unit was introduced via an aromatic nucleophilic substitution. Notably, benefiting from the easy identification of C≡N, C═O, COOMe in IR spectrum, we were able to confirm the conversion of step 3, 4, 5 via IR conveniently. The final target was freed from solid matrix in the treatment of TFA-CH₂Cl₂ (1:10, v/v) at room temperature. The overall yield of solid phase route is 70%. Additionally, benefitting from solid phase synthesis, flash chromatography after reaction can be avoided.

It was found that the translation to SPS-batch synthesis was straightforward, and no modification was required for the protecting, S_{N}2, hydrazine condensation, and cleavage steps. Further advantageously, the Claisen condensation in SPS was conducted at room temperature for the flow synthesis, which was different from the conditions used in the solution-batch synthesis (−78° C.). However, the Thorpe reaction occurred at elevated temperatures, which consumed the starting nitrile, and excess MeCN and LDA reagents were required to ensure a full conversion. The optimal conditions for the S_{N}Ar in solution-phase synthesis gave less than 30% conversion in SPS. It is suspected that the low reactivity may be caused by the limited swelling of the polymer resin in the DMSO solvent, which may limit substrate diffusion into the matrix. Using 1,4-dioxane as a co-solvent, which allowed more pronounced resin swelling, successfully promoted the S_{N}Ar to full conversion. The purification was done by a simple filtration between each step and a final crystallization from methanol and ethyl acetate. This SPS-batch six-step synthesis afforded pure prexasertib TFA salt in 53% overall yield.

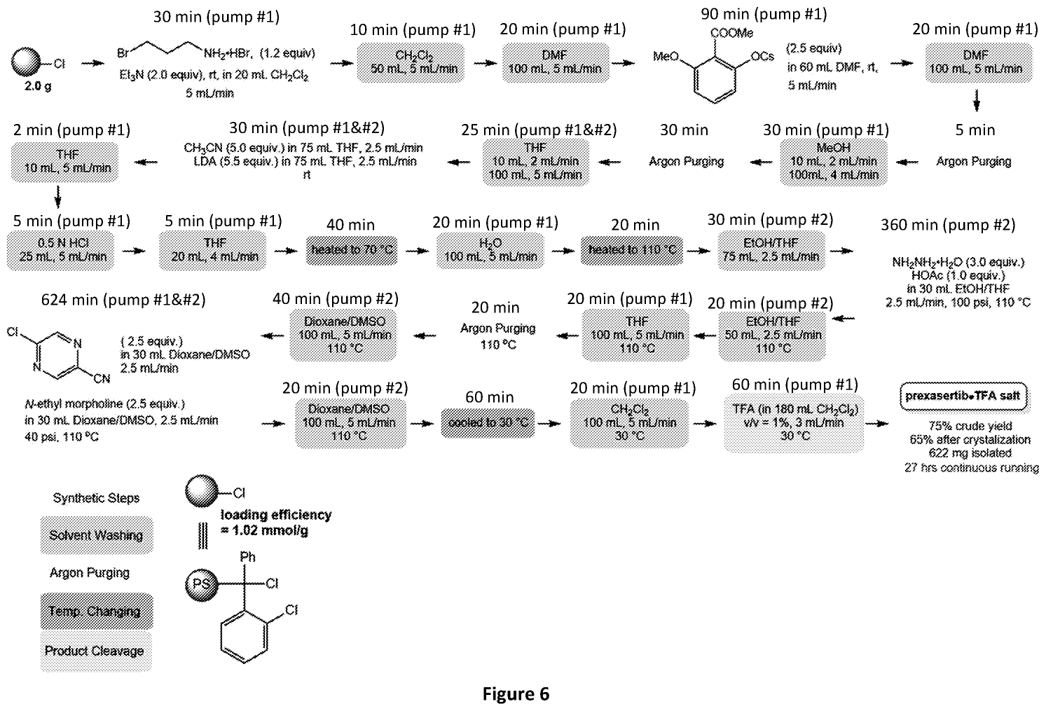

The SPS conditions can be transferred to the SPS-flow synthesis by incorporating solvent washing steps (FIG. 6). An example of the procedure is illustrated in FIG. 6. Circulating flow was applied for transformations requiring long reaction time (≥1 hour, including the protection, S_{N}2, hydrazine condensation, and S_{N}Ar steps). Direct one-way flow was employed for the Claisen condensation and the final cleavage step. Starting with 2-Cl trityl chloride resin, after 27 hours continuous running, 75% of crude overall yield was obtained, after recrystallization, a 65% isolated yield was obtained. Worth noting that finishing the reaction of 3rd step, Claisen condensation, within 30 minutes was necessary, since self-condensation of CH₃CN is more favorable at room temperature than below 0° C. Additionally, washing procedures and argon purging can further be included between each major synthetic step.

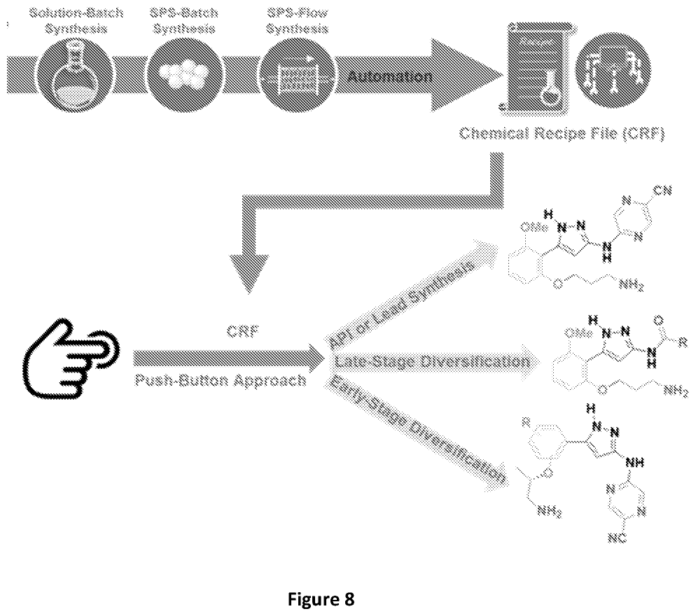

Argon purging can be used to improve the washing efficiency and to mitigate clogging risks associated with the Claisen condensation, which involves the use of LDA (FIG. 8). A CRF can therefore be established as illustrated in FIG. 8. Notably, LDA, which is an extremely moisture-sensitive reagent, was successfully incorporated in the middle of this multistep synthesis. This is challenging in solution-phase continuous-flow multistep syntheses due to reagent/solvent incompatibility. An automated push-button synthesis based on the CRF was achieved with LabVIEW™ programming, which specifies the process modules, the fluid and argon paths, the location of stock solutions and solvents, the flow rates, and the temperature. A fully automated 32-hour continuous synthesis with two grams of resin afforded 635 mg of prexasertib as the TFA salt (65% yield) after crystallization. HPLC analysis showed that the purity of the product was >99.9%. The digital defined CRF and the standardized reactor system allow the storage of this API synthesis electronically for implementation on demand.

A further advantage of the SPS-flow-based CRF is the versatility in very quickly synthesising a library of derivatives, which can be adapted to different types of chemistry, and allows each synthetic step to be considered independently that can in principle be replaced without disturbing the other steps. The system allows easily switching from one molecule to another, which makes it transferable between different target molecules, reducing the time and labor cost. Any change of reagents or solvents at any stage would not interfere with the following step. This enables not only late- but also early-stage diversification in an automated manner, which is particularly useful for targeted de novo design of drug molecules where SAR studies based on initial core structures are instructive for targeted selectivity profiles.

To showcase this capability, a library of twenty-three prexasertib derivatives was prepared by changing only one step in the six-step CRF including both late- and early-stage diversification. To ensure the applicability of the system, a study have been carried out with a set of 7 types of reactions and more than 25 different molecules at gram-scale synthesis. These reactions and derivatives are shown below.

Examples of derivatives that can be synthesized which are late-stage diversification:

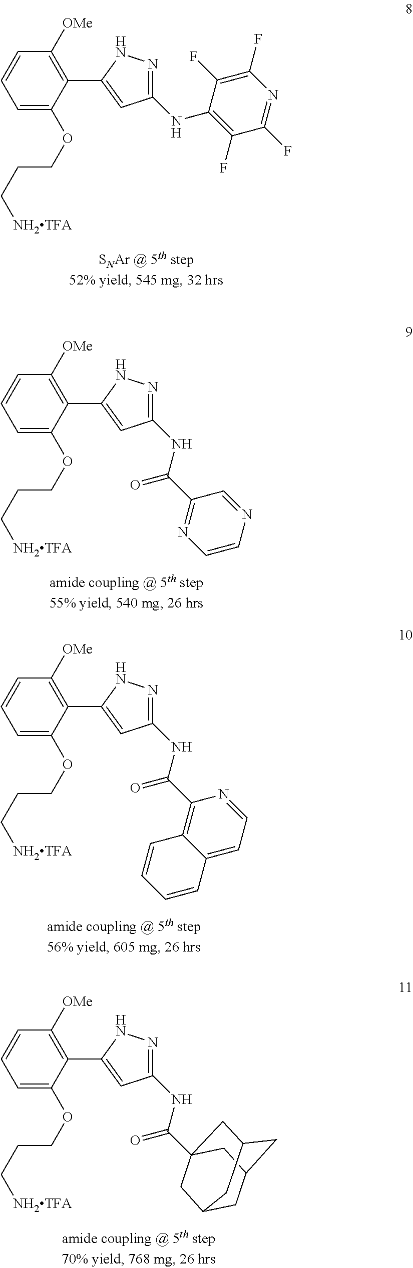

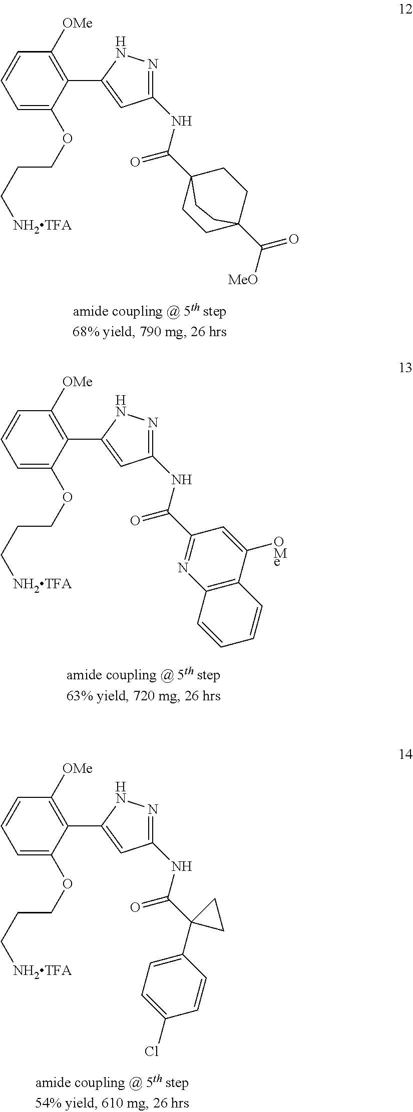

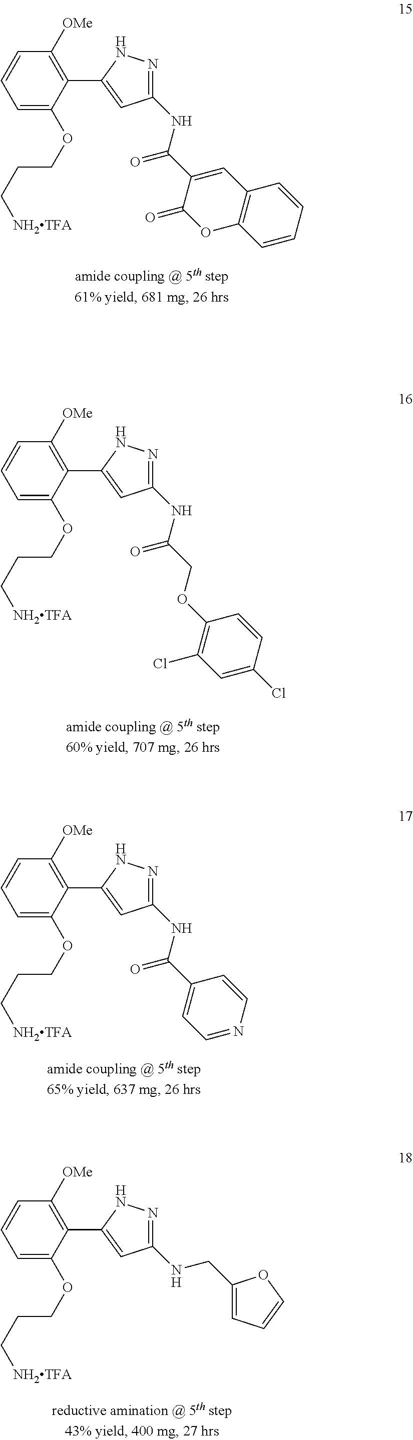

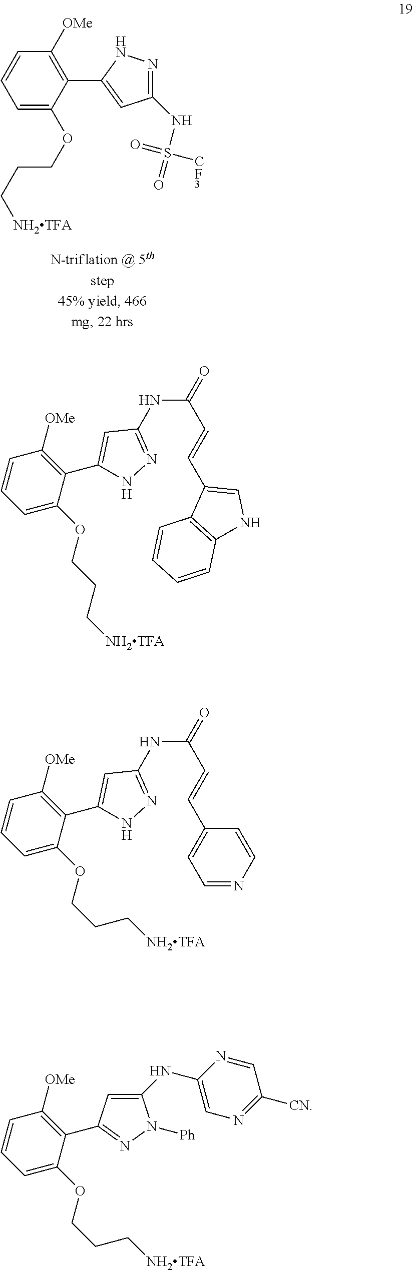

Examples of derivatives that can be synthesized which are early-state diversification:

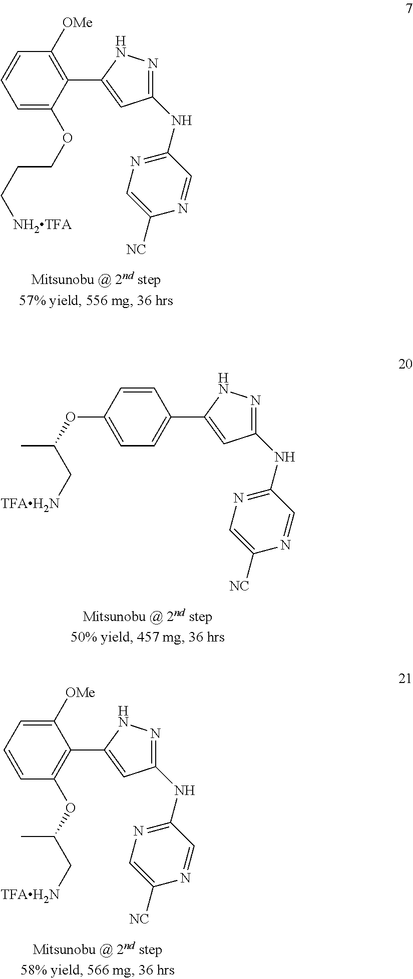

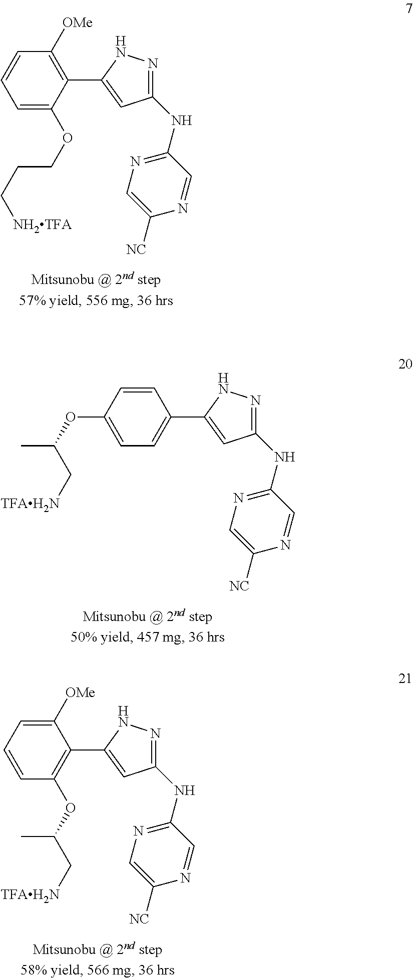

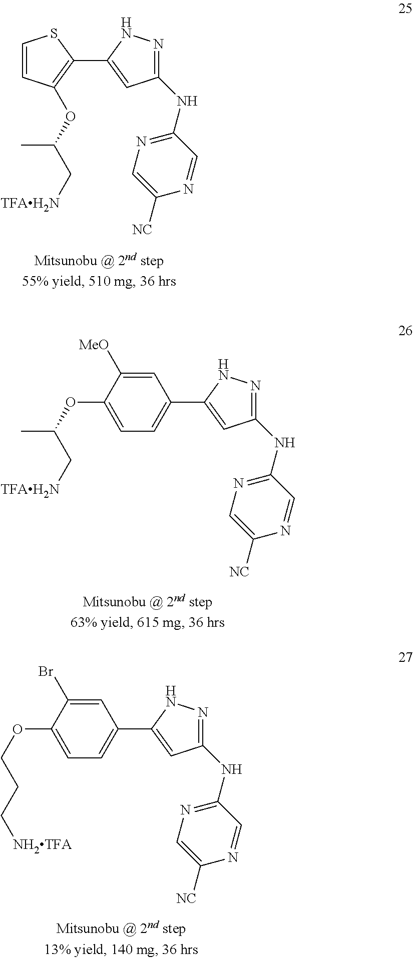

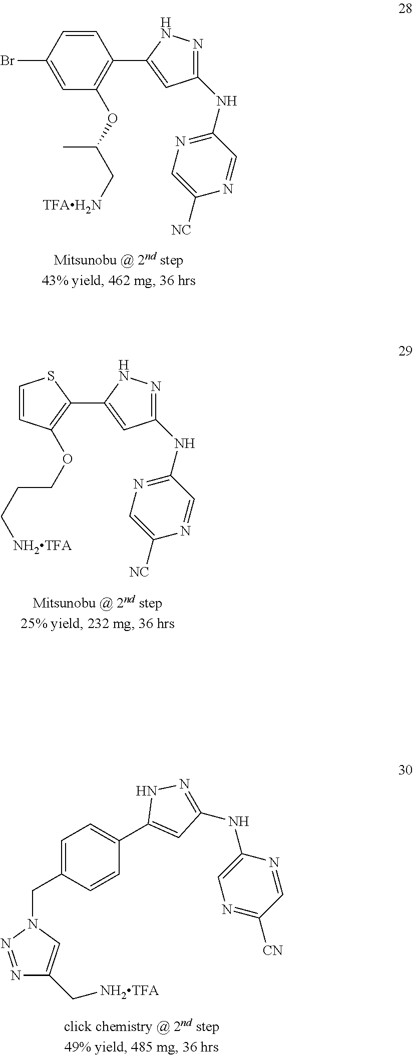

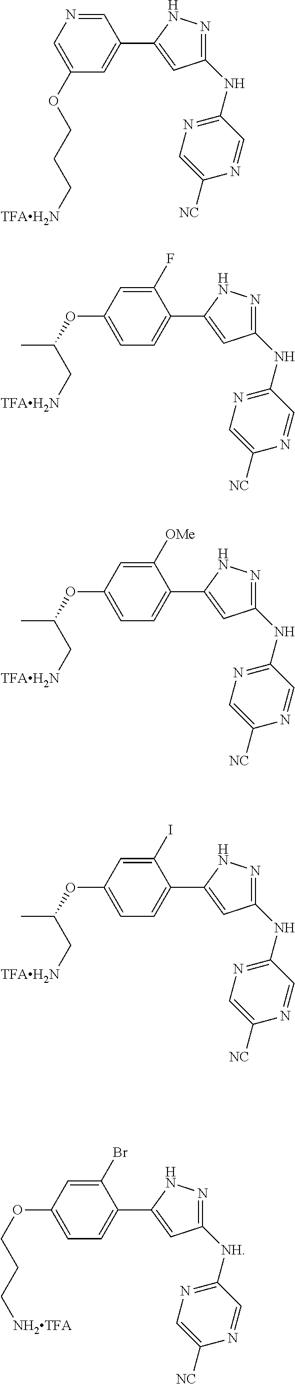

By exchanging 2-chloro-5-cyanopyrazine for pentafluoropyridine in the S_{N}Ar reaction but otherwise following the same CRF, prexasertib analog 8 was prepared in 52% isolated yield. Amide derivatives (9-17) were then accomplished in generally good yields by simply replacing the S_{N}Ar reaction with an amide coupling in the CRF, without altering the other steps. A fast cleaning procedure taking less than two hours was established between the production of each derivative. By replacing the S_{N}Ar transformation with a two-step reductive amination or an N-triflation transformation, 18 and 19 were produced in 43% and 45% yield, respectively. By conducting a Mitsunobu reaction in the CRF rather than an S_{N}2 transformation in the second step while keeping the other steps the same, the TFA salt of prexasertib (7) could still be generated in 57% yield. Different aryl and heteroaryl moieties could be incorporated into the scaffold by changing the stock reagents in the Mitsunobu reaction to realize early-stage diversification, furnishing products 20-29 in 13-63% isolated yields, with some of the products being obtained in enantiopure form. The last derivative, 30, was specifically chosen to demonstrate the versatility and diversity of this CRF strategy. A click reaction was used instead of the original S_{N}2 transformation to produce 30 in 49% isolated yield in a push-button approach followed by a simple off-line recrystallization.

By integrating SPS and continuous-flow synthesis, a simple and compact platform is developed for the on-demand automated synthesis of a target molecule and its derivatives.

Prexasertib was prepared in a six-step protocol in good yield as a proof-of-concept, and the generated CRF was modified for the synthesis of another twenty-three derivatives by simply switching the stock reagents or changing only one of the synthetic steps. The merits of this strategy include: 1) an extremely simple and compact platform compared to other automated multistep syntheses; this platform is versatile and can be used to access different targets without physical reconfiguration; 2) easy establishment of the CRF by translation of the solution-batch synthesis to SPS-flow synthesis; 3) The synthesis of a target molecule can be stored infinitely as a CRF for implementation on demand; 4) capability for derivative library synthesis by changing the stock reagents; and 5) versatility based on the incorporation or modification of one synthetic step without influencing the other steps in the CRF, thus significantly expanding the structural scope for both late- and early-stage diversification.

It is believed that the present invention allows for the realization of pharmaceutical production in response to sudden changes in demand, and will dramatically accelerate the drug development process. The present invention advantageously solves or at least ameliorates the desire for process intensification (e.g., to decrease overall reaction time), by at least combining reagent preparation, synthesis, and downstream purification and formulation into a single, compact unit. The present invention can also be linearly scaled for large-scale production, and parallel assembly of two SPS-flow column reactors or a solution-based continuous-flow system with a SPS-flow column reactor for convergent synthesis.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of synthesising a compound, including:
a) providing a solid support in fluid communication with a computer-controlled flow system;
b) covalently bonding a first precursor to the solid support for forming an extension thereon;
c) performing at least one reaction, the reaction including:
i) conditioning the extension with a first solvent;
ii) reacting a first reactant with the extension for synthesising the compound on the solid support; and
d) cleaving the compound from the solid support;
wherein the first precursor, the first solvent and the first reactant are deliverable to the solid support via the computer-controlled flow system.

2. The method according to claim 1, wherein the first reactant is provided in the first solvent.

3. The method according to claim 1, further including a step (ciii) after step (cii) of washing the compound with the first solvent.

4. The method according to claim 1, further including a step of purging with air or gas before the conditioning step (ci).

5. The method according to claim 1, further including a step of purging with air or gas after the washing step (ciii).

6. The method according to claim 1, wherein the solid support is a 2-chlorotrityl chloride resin.

7. The method according to claim 1, further including a step of altering a temperature of the reaction.

8. The method according to claim 1, wherein the first precursor comprises an amino moiety.

9. The method according to claim 1, wherein the at least one reaction is selected from a SN₂ reaction, hydrazine condensation, Thorpe reaction, Claisen condensation, S_{N}Ar reaction, amide coupling, N-triflation, reductive amination, phenyl-hydrazine cyclization, pyazole cyclization, click chemistry, or Mitsunobu reaction.

10. The method according to any one of claim 1, wherein the first precursor further comprises a second moiety selected from the group consisting of halide (except fluoride), tosylate, amino, carboxyl, carbonyl, triflate, aryl, alkynyl, azidyl, alkenyl, tetrazinyl, tetrazolyl, hydroxyl, hydrazoic acid, imide, thiophenol, sulphonamide, arylsulfonylhyrazine, hydrazine, or cyanoalkylacyl.

11. The method according to claim 1, wherein the compound is cleaved using trifluoroacetic acid.

12. A method of synthesising prexasertib, derivatives, salts, solvates or stereoisomers thereof, comprising:
a) providing a 2-chlorotrityl chloride resin in communication with a computer-controlled flow system;
b) covalently bonding a compound of Formula (I) to the 2-chlorotrityl chloride resin for forming an extension thereon, the compound of Formula (I) is:
T-M-NHR₁ (i)
wherein M is optionally substituted alkyl;
T is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₁ is selected from H, optionally substituted alkyl, optionally substituted alkenyl;
c) performing a first reaction, the first reaction including:
i) conditioning the extension with a first solvent;
ii) reacting a compound of Formula (II) with the extension for synthesising a first intermediate on the solid support, the compound of Formula (II) is: 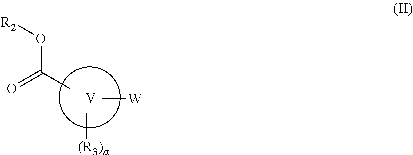
wherein V is selected from optionally substituted aryl or optionally substituted heteroaryl;
W is selected from halide (except fluoride), tosylate, hydroxyl, carboxyl, hydrazoic acid, imide, phenol, thiophenol, sulphonamide, arylsulfonylhyrazine, alkynyl, azidyl, alkenyl, tetrazinyl, or tetrazolyl;
R₂ is optionally substituted alkyl;
R₃ is independently selected from halide, optionally substituted alkyl, optionally substituted alkenyl, optionally substituted alkoxy;
a is an integer selected from 0, 1, 2 or 3;
d) performing a second reaction, the second reaction including:
i) conditioning the first intermediate with a second solvent;
ii) reacting CH₃CN and lithium diisopropylamide with the first intermediate for synthesising a second intermediate on the solid support;
e) performing a third reaction, the third reaction including:
i) conditioning the second intermediate with a third solvent;
ii) reacting NH₂NH₂ or NH₂NHPh with the second intermediate for synthesising a third intermediate on the solid support;
f) performing a fourth reaction, the fourth reaction including:
i) conditioning the third intermediate with a fourth solvent;
ii) reacting a compound of Formula (III) with the third intermediate for synthesising a fourth intermediate on the solid support, the compound of Formula (III) is: 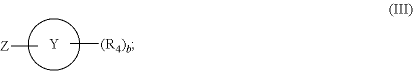
Wherein Y is selected from optionally substituted aryl, optionally substituted heteroaryl, optionally substituted cycloalkyl, or optionally substituted heterocycloalkyl;
Z is selected from carboxyl, carbonyl, alkenylacyl, triflate, aryl, alkyl (substituted with carboxyl, carbonyl or alkenylacyl), or alkoxy (substituted with carboxyl, carbonyl or alkenylacyl);
R₄ is selected from halo, cyano, optionally substituted alkoxy, optionally substituted alkyl, or optionally substituted alkenyl;
b is an integer selected from 0, 1, 2, or 3; and
g) cleaving the compound from the solid support;
wherein the compounds of Formula (I), (II), (II), CH₃CN, lithium diisopropylamide, NH₂NH₂ and solvents are deliverable to the 2-chlorotrityl chloride resin via the computer-controlled flow system.

13. The method according to claim 12, wherein when the synthesised compound is prexasertib, a yield is at least 60% and/or a purity is at least 99%.

14. (canceled)

15. A system for synthesising a compound, including:
a) a flow system;
b) a solid support in fluid communication with the flow system;
b) a first precursor covalently bonded to the solid support for forming an extension thereon;
c) at least a first solvent for conditioning the extension;
d) at least a first reactant for performing at least one reaction on the extension to synthesise the compound; and
d) a controller configured to regulate the flow system for flowing the first precursor, the first solvent and the first reactant to the solid support.

16. The system according to claim 15, wherein the solid support is contained in a reaction vessel; and wherein the flow system is connected to the reaction vessel for fluid communication with an interior thereof.

17. The system according to claim 15, wherein the flow system includes at least one multi-port input valve in communication with the controller, and wherein the controller is configured to actuate the at least one multi-port input valve for selective flow of the first precursor, the first solvent or the first reactant into the reaction vessel.

18. The system according to claim 15, wherein the flow system includes a multi-port output valve, and wherein the controller is configured to actuate the multi-port output valve for selective flow of output fluid from the reaction vessel to one of a plurality of output vessels.

19. The system according to claim 15, wherein the flow system includes a pressure regulator for regulating pressure inside the reaction vessel.

20. The system according to claim 15, further including at least one heating element in thermal communication with the reaction vessel, wherein the controller is configured to alter the temperature of the reaction.

21. The system according to claim 15, further including a circulative flow path for allowing the flow to return to a starting bottle and/or a one-way flow path for allowing the flow to elute to a waste bottle.
